(19) United States
(12) Patent Application Publication
(10) Pub. No.: US 2026/0199604 A1
(43) Pub. Date: Jul. 16, 2026

(54) PLUNGER FOR REDUCING THE DEAD VOLUME OF A SYRINGE

(71) Applicant: Transcoject GmbH, Neumünster (DE)

(72) Inventor: Jochen HEINZ, Flintbek (DE)

(21) Appl. No.: 19/136,411

(22) PCT Filed: Dec. 6, 2023
(86) PCT No.: PCT/EP2023/084600

§ 371 Date: Jun. 6, 2025

(30) Foreign Application Priority Data
Dec. 7, 2022 (EP) 22212000.8

(51) Int. Cl. A61M 5/315; A61M 5/34

(52) U.S. Cl. CPC A61M 5/31513; A61M 5/31501; A61M 5/31515; A61M 5/347; A61M 2005/31516

(57) ABSTRACT

A syringe (1) has a longitudinal portion (5), in which a piston (10) is guided in a movable manner, which closes the syringe cylinder (5) at one end and which is movable by means of a piston rod (9). On the distal side, a connection (4) is provided for fitting an injection needle (11) at the other end of the syringe cylinder (5). For reducing the dead volume, the piston (10) is displaceable beyond the distal end of the connection (4).

### CROSS REFERENCE TO RELATED APPLICATIONS

This application is a United States National Phase Application of International Application PCT/EP2023/084600, filed Dec. 6, 2023, and claims the benefit of priority under 35 U.S.C. § 119 of European Application 22212000.8, filed Dec. 7, 2022, the entire contents of which are incorporated herein by reference.

### TECHNICAL FIELD

The invention relates to an application vessel, in particular a syringe with a longitudinal portion, in which a piston is guided in a movable manner, which closes the application vessel at one end and which is movable by means of a piston rod. The application vessel has a connection for fitting an injection needle at the other end, and can be a presented application vessel or a vessel which is fillable before the application, as is usual in the case of syringes.

### BACKGROUND

With the increasing development of sensitive, highly effective, high cost or also limited-availability active ingredients for injection, which generally already take effect with a small dosage in the region of microlitres, a requirement exists for application vessels, in particular syringes, which on the one hand can dose in a very precise manner small quantities, also in the case of manual injection, and in which, on the other hand, as little active ingredient as possible remains unused in the application vessel and in the connected injection needle, which must not be used further and must be disposed of together with the vessel.

For the injection of small volumes, it is known to use disposable syringes which have a small internal diameter of e.g. 4.5 mm, are provided with a fine scale and are provided for the injecting of for example 1 ml. Included here in the prior art, for the reducing of the dead volume, is the equipping of these with a so-called displacement plug, which projects into the Luer cone, therefore into the connection of the application vessel, in particular of the syringe, and which displaces the liquid contained therein and dispenses it through the needle. For injection, however, a cannula must be fitted, wherein the dead volume of the cannula socket and of the needle remains in a constant manner. An optimizing of the dead volume which exceeds this is hardly possible with the use of the Luer standard which is introduced worldwide, however a deviation from this standard would lead to the syringe losing its compatibility with the available cannulas and hence ultimately also its general usability.

The syringes with integrated needle, which are able to be used alternatively, in which the needle does not have to be fitted, but rather is configured in one piece with the syringe, can indeed be constructed with a smaller dead volume, however the needle is not then exchangeable. In addition, the finest needs which are available today are only able to be connected to the syringe with difficulty in terms of manufacturing technology. The needle should also be changed after drawing up the medicament, in order to thus rule out damage to the needle tip, adhesions of medicaments, non-sterilities and suchlike. Drawing-up cannulas are also frequently not suitable for injection.

If, however, pre-filled syringe systems are used, then the above-mentioned problems can be largely avoided. For precise dosing, however, as small a diameter of the syringe as possible is required, which is to remain constant over the length of the syringe, wherein the syringe must not exceed a manageable length for receiving the required volume. The usual minimum filling volume today in glass syringes is 0.5 ml, the glass tube of which has a minimum internal diameter of 4.5 mm and which are available with a Luer- or Luer-lock connection, but also with an integrated needle. Glass syringes have production-related disadvantages here with regard to dead volume and dosing accuracy, compared to plastic syringes, as the latter can be manufactured more precisely with regard to internal diameter and the contour of the top of the syringe. With regard to the dimensions, however, plastic syringes are also oriented to the conventional dimensions for glass syringes, owing to the industrial standards which have become established.

In the case of pre-filled syringes with integrated needle, the needle tip must be closed in a sealed and sterile manner, which generally takes place by a needle cap. In particular in the case of very thin needle, as are necessary e.g. for intraocular injection, the risk exists here that the needle bends or is damaged, so that the entire syringe, together with contents, becomes unusable.

Furthermore, carpules made of glass or of plastic belong to the prior art, which are integrated into a motor-driven feed for precise control, which feed permits a very precise and reproducible dosability. However, the residual emptying capacity in carpules is rather unfavourable, in particular when they are closed by a septum which is, if applicable, pierced multiple times by a needle of the syringe which is to be filled.

### SUMMARY

The invention is based on the problem of configuring an application vessel, in particular a syringe of the type mentioned in the introduction, so that the previously mentioned problems are overcome, in particular the dead volume on injecting is reduced.

This problem is solved according to the invention by an application vessel with the features indicated. Advantageous configurations of the invention will emerge from the claims, from the following description and from the drawings.

The application vessel according to the invention, in particular the syringe according to the invention, has a longitudinal portion in which a piston is guided in a movable manner, which closes the application vessel at one end and which is movable by means of a piston rod. The application vessel has a connection for fitting an injection needle at the other end of the application vessel. According to the invention, the piston is displaceable for reducing the dead volume beyond the distal end of the connection. It is to be noted at this point that the terms “distal” and “proximal” refer herein to the person using the application vessel and not to the patient to whom the content of the application vessel is applied.

The basic idea of the solution according to the invention is to configure an application vessel with a connection for fitting an injection needle, and with a piston guided movably therein, so that the piston is displaceable beyond the distal end of the connection and thus to at least partially fill by the piston the dead volume otherwise present in the region of the counter-connection piece with the injection needle, in order to dispense the agent, situated in the application vessel, as completely as possible through the injection needle.

An application vessel in terms of the invention is typically a syringe cylinder, but can also be a different vessel for the storing and dispensing of a medicament or of another medicinal agent. The cross-sectional shape of the longitudinal portion in which a piston is guided in a movable manner is, however, preferably not necessarily circular here, it can also be oval or triangular or respectively polygonal and preferably with rounded corners. The piston, which does not necessarily have an identical cross-sectional shape, but has a shape which is at least adapted thereto, is arranged here displaceably but in a sealing manner within the longitudinal portion, so that the vessel, in particular the syringe cylinder, is closed at one end by the piston, and has at the other end the connection for fitting an injection needle with counter-connection piece or another line counter-connection. After travelling over the distal end of the connection on the vessel side, the piston can dip at least into the counter-connection piece with the injection needle or into the other line counter-connection, in order to at least partially displace the dead volume situated there. Advantageously, the arrangement is such that the piston completely displaces the dead volume in the counter-connection piece up to the needle.

Advantageously, this takes place structurally in that the internal cross-section of the longitudinal portion, in which the piston is guided in a movable manner, continues into the internal cross-section of the connection or is smaller than the latter. Preferably, the inner wall of the longitudinal portion continues flush, preferably in a step-free manner, into the inner wall of the connection. It shall be understood that in the case of plastic vessels, in particular in the case of plastic syringes which are produced by injection moulding, a certain conicity of this inner wall always exists, which is necessary for the forming of the workpiece. This conicity is negligible in so far as the previously mentioned consistent cross-section is concerned which is essential for the invention.

The configuration of the connection, in particular of the syringe connection as a Luer- or Luer-lock connection, is particularly advantageous since, as already mentioned in the introduction, this ensures a high degree of compatibility of the application vessel according to the invention with existing cannulas or line connections. With the use of a Luer- or Luer-lock connection, it is expedient to form the piston and the longitudinal portion of the application vessel surrounding the latter so as to be circular in cross-section and to configure the internal diameter of the cylinder smaller than the external diameter of the (male) Luer cone, so that the piston can displace as completely as possible the dead volume formed in the (female) counter-connection piece on the Luer cone.

When the inner wall of the longitudinal portion of the application vessel continues into the inner wall of the Luer cone of the Luer- or Luer-lock connection, a high displacement volume is produced within the fitted Luer- or Luer-lock counter-connection piece.

According to an advantageous further development of the invention, this displacement volume can be further increased if at least one portion at the distal end of the piston is configured in a pliant-elastic manner at least transversely to its displacement direction, in such a way that after displacing of the distal piston end, it expands radially beyond the distal end of the connection. With such a configuration, practically the entire volume of the counter-connection piece can be filled up to the needle or respectively to the narrow recess leading to the needle.

Then practically only the internal volume of the needle and of the channel leading thereto in the counter-connection piece remains as dead volume.

Pliant-elastic in the context of the invention is to be understood to mean pliant or respectively flexible materials (elastomers) which have a modulus of elasticity <700 MPa. Hard-elastic in the context of the present invention, on the other hand, is to be understood to mean rigid and semi-rigid materials, as are defined in the standard ISO 80369. Here, rigid materials have a flexural-or tensile-elasticity modulus, which is >3,433 MPa, and semi-rigid materials have such a modulus between 700 and 3,433 MPa.

Apart from the conicity necessary if applicable for the demoulding of the syringe, it is, however, advantageous to configure the longitudinal portion in which the piston is movable with a uniform internal cross-section, which continues up into the connection. Preferably, this longitudinal portion is configured in a cylindrical manner and has a circular cross-section.

Optionally, with an injection needle fitted on the connection in distal direction from the distal end of the connection, a dead space can extend with an axial dead space length and with a radial dead space internal cross-section, wherein the piston is displaceable into the dead space. This dead space can result through the standardized configuration of the counter-connection piece of the injection needle with a female Luer- or Luer-lock connection, when it is fitted onto the male Luer- or Luer-Lock connection of the application vessel.

Optionally, the piston can have an axial length which is greater than the axial dead space length. This has the advantage that a proximal part of the piston remains in the connection in a sealing manner, and thus prevents a backflow into the longitudinal portion.

Optionally, the piston rod can be sufficiently long so that the piston in the dead space strikes axially against a counter-connection piece of the injection needle and is thereby compressed axially in the dead space plastically and/or elastically, and widens in the dead space radially onto the dead space internal cross-section. The dead space is then filled almost entirely by the piston through manual compressing of the piston, so that practically only the internal volume of the needle and of the channel leading there remains in the counter-connection piece as remaining dead volume. Through the axial compressing of the piston, a proximal rear edge of the piston can be pressed beyond the distal end of the connection, so that the proximal rear edge of the piston engages behind an axial end face of the connection with radial widening in the dead space.

Optionally, the dead space internal cross-section, at least at the proximal end of the dead space, can be greater than an internal cross-section of the longitudinal portion which continues up into the connection, wherein at least a part of the piston, for example an exterior sealing lip of the piston, is configured at least transversely to its displacement direction in a pliant-elastic manner such that it widens in the dead space radially onto the dead space internal cross-section. The dead space is then filled almost entirely by the piston in that it is radially compressed in the internal cross-section of the longitudinal portion and independently widens radially in the dead space in an elastic manner, so that practically only the internal volume of the needle and of the channel leading thereto in the counter-connection piece remains as residual dead volume. The axial length of the piston can correspond approximately to the dead space length. Thereby, a proximal rear edge of the piston can be pressed beyond the distal end of the connection, so that the proximal rear edge of the piston engages behind an axial end face of the connection with radial widening in the dead space.

In order to prevent the piston from exerting, in extended position, too great a force onto the counter-connection piece in axial direction, it is advantageous to limit the axial extension path of the piston in distal direction. The limiting takes place here expediently so that the pliant-elastic part of the piston, by running into the counter-connection piece, practically entirely fills the dead volume situated therein, without, however, exerting an unduly high force distally in axial direction, which could jeopardize the connection between connection piece and counter-connection piece.

Advantageously, such a limit can be formed by a detent arrangement which is preferably formed between piston and inner wall of the application vessel, or between piston rod and the application vessel. Instead of a detent position, a stop can also be provided, which limits the distal extension path of the piston.

In addition to the limiting of the extension path, it can be advantageous to provide one or more detent arrangements, for example with a detent position in which the piston closes the application vessel but is not yet moved for the dosed dispensing of the vessel content, and/or a detent position in which the distal piston end has reached the distal end of the Luer connection.

It is particularly advantageous if the application vessel, when it is configured as a syringe, has at the proximal end a finger rest which forms a stop for a thumb rest arranged at the end of the piston rod. The handling for manual discharging is then particularly good, and operating errors are practically ruled out.

The configuration according to the invention is suitable in particular, but not only, for syringes with a small discharge volume of for example 0.3 ml. It is excellently suitable here as a pre-filled application vessel, wherein the Luer- of Luer-lock connection is closed in a sealed manner by a terminal cap which is removed immediately before use and is replaced by an injection needle provided with a Luer- or respectively Luer-lock counter-connection.

It is particularly advantageous if the cap closing off the application vessel is configured so that, at the same time, is blocks the retraction movement of the piston, e.g. covers the thumb rest, or else a separate protective cap is provided, which is fitted on the proximal side of the application vessel and which shields the piston rod, in particular the thumb rest of the piston rod, against an actuation. This cap is to be removed before use.

Basically, the configuration according to the invention can take place with a plurality of application vessels which are suitable for this; it is particularly advantageous in syringes, irrespective of whether the syringe body is formed from glass or from plastic. Particularly advantageously, however, the invention is to be realized with a syringe body made of plastic. Advantageously, the configuration according to the invention is used with a pre-filled syringe.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a syringe with Luer-lock connection in longitudinal section;

FIG. 2 is a syringe with Luer connection and fitted counter-connection piece with needle in longitudinal section;

FIG. 3 is an alternative configuration in illustration according to FIG. 2;

FIG. 4 is in a highly enlarged longitudinal section view, a Luer-lock connection with inner cone, on the vessel side;

FIG. 5 is a schematic view showing a syringe with piston in five different positions;

FIG. 6 is a syringe with fitted injection needle and proximal protective cap; and

FIG. 7 is the syringe according to FIG. 6 with fitted distal protective cap.

### DESCRIPTION OF PREFERRED EMBODIMENTS

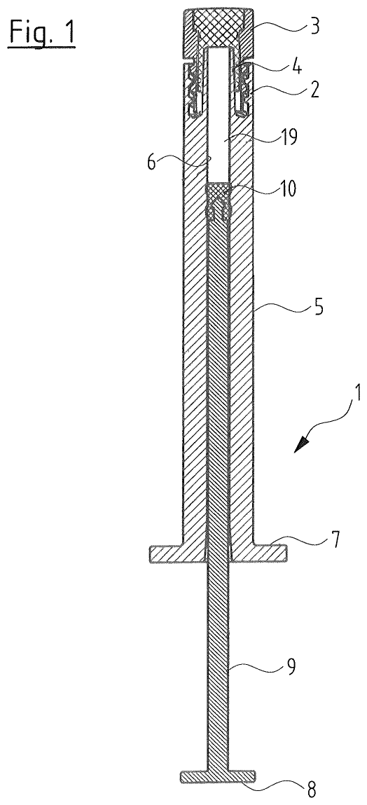

Referring to the drawings, the syringe 1 illustrated in FIG. 1 concerns a pre-filled syringe with a Luer-lock connection on the distal side, which closes off in a sealing manner the distal end of the syringe 1, in particular of the Luer cone 4, by a protective cap 3 sitting thereon. The syringe has a syringe cylinder 5, the cylindrical inner wall 6 of which continues up into the Luer cone 4, so that a continuous cylindrical inner wall is formed, which extends through the entire syringe 1. At the proximal end of the syringe cylinder 5, a finger rest 7 is formed which, in connection with a thumb rest 8 at the proximal end of a piston rod 9, is provided for the manual dispensing of the agent situated in the syringe cylinder. The actual piston 10, which is arranged in an axially movable manner within the syringe cylinder 5 and seals the latter on the proximal side, sits at the end of the piston rod 9. The syringe cylinder 5 and piston rod 9 are made from hard-elastic plastic, whereas the piston 10 is made from pliant-elastic plastic, just as the interior of the protective cap, which closes off the Luer cone 4 in a sealing manner. The elasticity of the material here is such that the piston 10, on travelling over the distal end of the Luer cone 4, widens radially in order to fill the dead volume, existing there, in the female counter-connection piece of the Luer cone

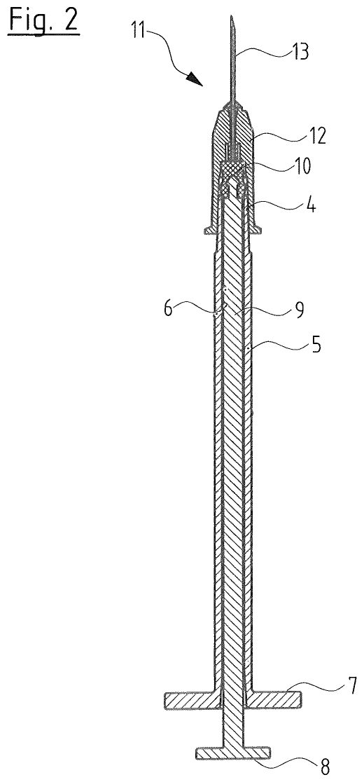

The axial dimensioning of piston rod 9 with piston 10 and the syringe cylinder 5 with Luer cone 4 is not to scale in FIG. 1 and is so that the piston 10 can be pushed by means of the piston rod 9, after removal of the protective cap 3, so far in distal direction that it projects on the distal side of the Luer cone 4, in order to fill the dead space formed in this region in the case of a fitted counter-connection piece, as is the case in the illustration of the syringe according to FIG. 2. In the case of the syringe according to FIG. 2, a Luer cone 4 is formed at the end of the syringe cylinder 5, on which an injection needle 11 is fitted, which is fastened on the Luer cone 4 with its counter-connection piece 12. There, the piston rod 9 is pushed with the piston 10, fastened thereon on the distal side, beyond the distal end of the cone 4 up into the counter-connection piece 12, and namely up to a shoulder in which the counter-connection piece 12 tapers to receive the needle 13. In this position, the syringe content is entirely dispensed and only a minuscule remaining cavity, formed by the needle 13, is still filled.

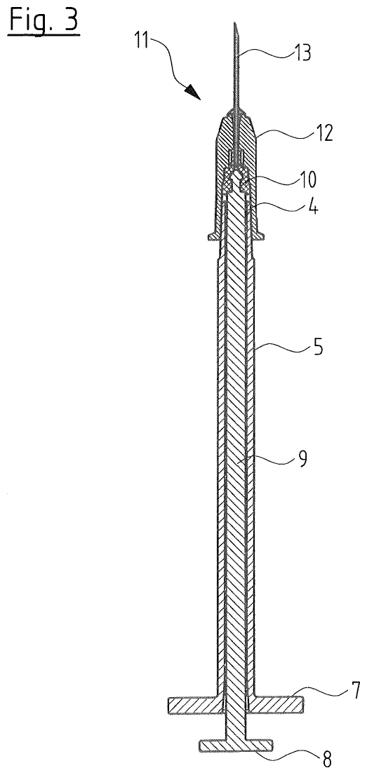

In the variant embodiment illustrated by means of FIG. 3, the counter-connection piece 12 is configured in a tapering manner in the region 14 projecting over the Luer cone 4, so that the pliant-elastic piston 10 can fill the dead volume there practically entirely.

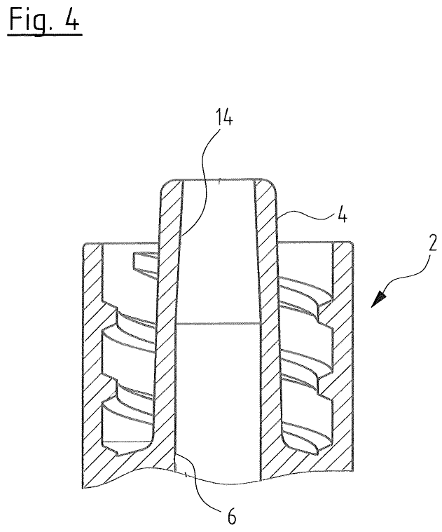

The sectional illustration according to FIG. 4 shows a configuration of the Luer-lock connection 2 on the syringe side, in which the cylindrical inner wall 6 continues in the region of the Luer cone 4 into a region 14 tapering conically in distal direction. In this region 14 the pliant-elastic piston 10 is compressed radially during advancing, whereby the slide resistance increases, so that the user senses that the piston 10 has just now arrived at the end of the Luer cone 4 and then only the short distance for removing the dead volume in the counter-connection piece 12 is to be travelled over. At the same time, the radial compressing leads to the piston 10 widening better radially the other side of Luer cone 4.

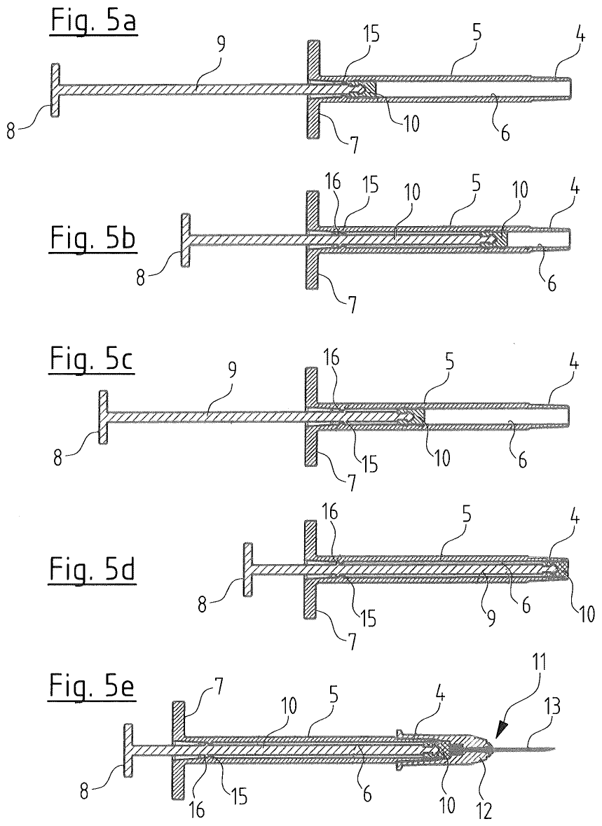

The handling of such a syringe 1 can be simplified by suitable detent arrangements, as is illustrated with the aid of FIG. 5. The syringe illustrated in FIG. 5a has a circumferential detent nose 15 on the cylindrical inner wall 6 close to the proximal end, which is arranged so that the piston rod 9 can not be removed entirely from the syringe with the piston 10, but rather only up to the drawing up of the maximum possible volume. In the illustration according to FIG. 5b, in addition to the circumferential detent nose 15, a circumferential detent nose 16, interacting therewith, is provided on the piston 10, which marks a particular position of the piston 10, for example a previously established filling volume.

The configuration of the detent noses 15 and 16 is expediently such that these are able to be travelled over by elastic deformation after overcoming a resistance. In the syringe illustrated with the aid of FIG. 5c, such a detent position is provided with an approximately half-retracted piston 10. The detent arrangement illustrated with the aid of FIG. 5d marks the position in which the piston 10 has reached the distal end of the Luer cone 4, therefore represents a resistance which signals to the operator that now after overcoming this resistance only the dead volume is to be filled in the counter-connection piece 12.

Finally, FIG. 5e shows an arrangement which can be configured as a detent arrangement, but also as a stop, depending on the radial extent of the circumferential detent noses 15 and 16. In the position illustrated with the aid of FIG. 5e, it is advantageous to provide a stop instead of the detent arrangement, as this is namely the end position in which the piston 10 has been moved out over the Luer cone 4 in a distal direction up into the counter-connection piece 12. It lies there against the shoulder in the counter-connection piece 12. By the stop, it can be effectively prevented that the axial force by the piston 10 onto the counter-connection piece 12 becomes unduly high and could jeopardize the secure seat of the counter-connection piece on the Luer cone 4.

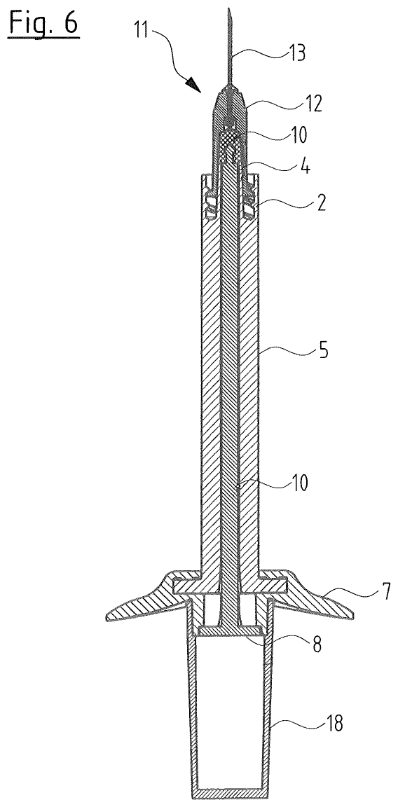

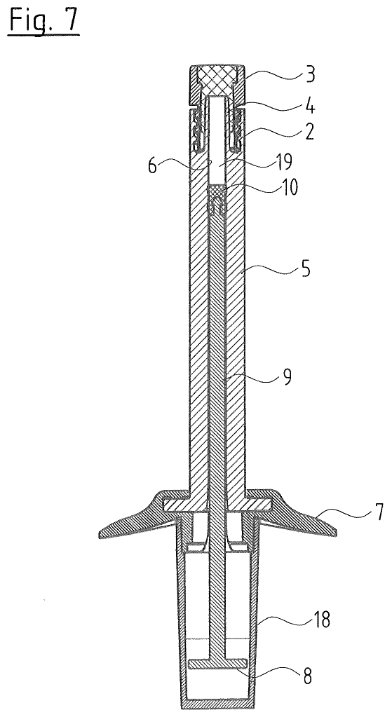

In the syringe illustrated according to FIG. 6, in which the piston 10 is situated in its distal end position, in which the piston 10 is situated in the counter-connection piece 12, the stop is formed by the thumb rest 8, which lies against the finger rest 7, mounted there as a separate component. This makes it clear that depending on the configuration of the piston-cylinder arrangement, the stop does not necessarily have to be provided between piston rod and cylinder, but can also be formed e.g. by the thumb rest 8 at the end of the piston rod 9 in connection with the finger rest 7. The finger rest 7 is, furthermore, lengthened in proximal direction by a cylindrical projection 17, which is provided for the fitting on of a proximal protective cap 18, which protects the proximal end of the piston rod 9 with the thumb rest 8 against an actuation. This proximal protective cap 8 is advantageous in particular in the case of a pre-filled syringe, as is illustrated with the aid of FIG. 7, in which the piston rod 9 is extended in proximal direction, and the volume 19 is formed between cylindrical inner wall 6, the piston 10 and the end of the Luer cone 4, which is closed by the protective cap 3 in a secure and sealing manner.

As the above embodiments clarify, individual features of the present invention can be advantageously applied on their own, but also in combination.

While specific embodiments of the invention have been shown and described in detail to illustrate the application of the principles of the invention, it will be understood that the invention may be embodied otherwise without departing from such principles.

### LIST OF REFERENCE NUMBERS

1 syringe
2 Luer-lock connection
3 protective cap
4 Luer cone
5 syringe cylinder
6 cylindrical inner wall
7 finger rest
8 thumb rest
9 piston rod
10 piston
11 injection needle
12 counter-connection piece
13 needle
14 conically tapering region
15 detent nose
16 detent nose
17 cylindrical projection
18 proximal protective cap
19 volume

1. An application vessel comprising:
a longitudinal portion;
a piston with a piston rod, piston the piston being guided in a movable manner in the longitudinal portion, and which piston closes the application vessel at one end and which is movable by means of the piston rod;
a connection for fitting an injection needle at another end of the application vessel, wherein the piston, for reducing a dead volume, is displaceable beyond a distal end of the connection, wherein the connection is configured as a Luer- or Luer-lock-connection, wherein a dead space extends, when the injection needle is fitted on the connection, with an axial dead space length and with a radial dead space internal cross-section in a distal direction from the distal end of the connection, wherein the piston is displaceable into the dead space, wherein the dead space internal cross-section is greater than an internal cross-section of the longitudinal portion, which continues on the distal side up into the connection, wherein at least a part of the piston is configured to be pliant-elastic at least transversely to a piston displacement direction such that the part of the piston widens in the dead space radially into the dead space internal cross-section.

2-3. (canceled)

4. The application vessel according to claim 1, wherein the longitudinal portion has a uniform cylindrical internal cross-section, which continues up into the connection.

5. (canceled)

6. The application vessel according to claim 1, wherein the piston has an axial length which is greater than the axial dead space length.

7. The application vessel according to claim 1, wherein the piston rod is sufficiently long so that the piston in the dead space strikes axially on a counter-connection piece of the injection needle and thereby is compressed axially in the dead space.

8. (canceled)

9. The application vessel according to claim 1, wherein the axial extension path of the piston is limited in the distal direction.

10. The application vessel according to claim 1, wherein at least one detent arrangement is provided between piston and an inner wall of the application vessel, or between the piston rod and the application vessel.

11. The application vessel according to claim 10, wherein a detent arrangement is provided with a detent position in which the piston has reached the distal end of the Luer- or Luer-Lock connection.

12. The application vessel according to claim 10, wherein a detent arrangement is provided with a detent position in which the piston closes the application vessel, but has not yet moved for the dosed dispensing of the vessel content.

13. The application vessel according to claim 1, wherein a stop is provided for the piston or the piston rod, which limits the travel path of the piston in the distal direction.

14. The application vessel according to claim 13, wherein the stop is arranged so that the stop limits the travel path of the piston before or up to reaching a shoulder of a canula socket of a canula fitted onto the Luer- or Luer-lock connection.

15. The application vessel according to claim 1, wherein at a proximal end of the application vessel, a finger rest is arranged, which forms a stop for a thumb rest arranged at the end of the piston rod.

16. The application vessel according to claim 1, wherein the application vessel is pre-filled and is provided with a cap (3) closing off the Luer- or Luer-lock connection.

17. The application vessel according to claim 15, further comprising a protective cap, which is fitted on a proximal side of the application vessel and which shields the thumb rest of the piston rod against an actuation.

18. The application vessel according to claim 1, wherein the application vessel consists of plastic and is configured as a plastic injection moulded part.

19. The application vessel according to claim 1, wherein the internal diameter of the syringe cylinder is smaller than the external diameter of a Luer cone of the connection.
